(19) United States
(12) Patent Application Publication
(10) Pub. No.: US 2021/0290167 A1
(43) Pub. Date: Sep. 23, 2021

(54) DEFORMATION MODEL FOR A TISSUE

(71) Applicant: Siemens Healthcare GmbH, Erlangen (DE)

(72) Inventor: Daniel Rinck, Forchheim (DE)

(73) Assignee: Siemens Healthcare GmbH, Erlangen (DE)

(21) Appl. No.: 17/205,381

(22) Filed: Mar. 18, 2021

(30) Foreign Application Priority Data
Mar. 19, 2020 (DE) 102020203526.1

(51) Int. Cl. A61B 5/00; A61B 90/00; A61B 5/055; G16H 30/40; G16H 30/20

(52) U.S. Cl. CPC A61B 5/70; A61B 90/36; A61B 5/7264; A61B 5/4806; A61B 2090/364; G16H 30/40; A61B 5/7246; G16H 30/20; A61B 5/055

(57) ABSTRACT

The disclosure relates to techniques for generating a deformation model for a tissue of an examination object in dependence on a positioning of the examination object. The technique may include provisioning of long-term MR data recorded in a first time period from a region of interest comprising the tissue of the examination object, an ascertainment of time-resolved first position data describing the tissue based on the long-term MR data, a provisioning of time-resolved second position data recorded in the first time period from at least two surface points of a surface of the examination object, and a determination of the deformation model for the tissue by correlating the first position data with the second position data.

### CROSS-REFERENCE TO RELATED APPLICATIONS

The present application claims the benefit of the filing date of German patent application no. DE 10 2020 203 526.1, filed on Mar. 19, 2020, the contents of which are incorporated herein by reference in their entirety.

### TECHNICAL FIELD

The disclosure relates to a method, a system, a magnetic resonance device, a computer program product and an electronically readable data carrier for generating a model of a tissue and for using the model.

### BACKGROUND

Knowledge of deformation of a tissue and/or an organ of an examination object due to a movement or repositioning of the examination object is, for example, relevant for an interventional examination. Similarly, such knowledge can be advantageous when registering different types of image data of the tissue, in particular if the examination object has a different positioning and/or is subject to movement during the recording of the image data.

### SUMMARY

The object underlying the disclosure is to generate a particularly precise deformation model for a tissue of an examination object dependent upon a positioning of the examination object. The object is achieved by the features of the embodiments as described herein and in the claims.

The method according to the disclosure for generating a deformation model for a tissue of an examination object dependent upon a positioning of the examination object may include the following method steps:

provisioning of long-term MR data recorded in a first time period from a region of interest comprising the tissue of the examination object;

ascertaining of time-resolved first position data describing the tissue based on the long-term MR data;

provisioning of time-resolved second position data recorded in the first time period from at least two surface points of a surface of the examination object; and

determining of the deformation model for the tissue by correlating the first position data with the second position data.

In a magnetic resonance device, the body of an examination object, e.g. a patient, to be examined is usually exposed with the aid of a main magnet to a relatively high main magnetic field, for example 1.5 or 3 or 7 tesla. In addition, gradient pulses are applied with the aid of a gradient coil unit. High frequency radio-frequency pulses, for example excitation pulses, are then emitted by means of suitable antenna devices via a radio-frequency antenna unit which leads to the nuclear spins of certain atoms excited to resonance by these radio-frequency pulses being tilted by a defined flip angle relative to the magnetic field lines of the main magnetic field. Upon relaxation of the nuclear spins, radio-frequency signals, known as magnetic resonance signals, are radiated and received by means of suitable radio-frequency antennas and then further processed. Finally, the desired image data can be reconstructed from the raw data acquired in this way. The region of the examination object within which the magnetic resonance signals are generated and from which the raw data is acquired is designated a region of interest. The region of interest is typically a sub-region of the examination object.

Therefore, a specific measurement requires the emission of a specific magnetic resonance control sequence (MR control sequence), also called a pulse sequence, consisting of a sequence of radio-frequency pulses, for example excitation pulses and refocusing pulses, as well as gradient pulses to be emitted suitably coordinated thereto on different gradient axes along different spatial directions. Temporally matching read-out windows are set which specify the time periods in which the induced magnetic resonance signals are acquired.

An examination object is typically a patient. The tissue is typically a functionally and/or organically and/or spatially self-contained sub-region of the examination object, typically arranged within the examination object. The tissue is typically an organ, such as, for example, a liver and/or a heart. A positioning of the examination object can also be designated the positional location of the examination object. In particular, the alignment of the extremities to the trunk and/or to one another can be characteristic of a positioning of the examination object. Positioning can be determined by the orientation of the examination object, e.g. a person's trunk and/or head, relative to the space and/or to a horizontal plane and/or an axis of the magnetic resonance device.

Long-term MR data is magnetic resonance data (MR data) recorded quasi-continuously within the first time period. Magnetic resonance data can comprise raw data and/or image data reconstructed therefrom, which was recorded by means of a magnetic resonance device from an examination object. The first time period may have a duration of any suitable length of time, such as for example at least one hour, at least two hours, at least three hours, etc. As additional examples, the first time period can also have an even longer duration of at least four or five or six hours.

Quasi-continuous recording of MR data within a time period, e.g. within the first time period, is characterized in that MR data is acquired from the region of interest at different time points within the time period, e.g. within the first time period. The quasi-continuous recording of MR data within a time period may take place at regular time intervals. The time intervals between two successive quasi-continuous recordings may be any suitable length of time, such as for instance shorter than 15 minutes, shorter than 10 minutes, shorter than 5 minutes, etc. The quasi-continuous acquisition of the MR data, e.g. the raw data, may take place by means of the same MR control sequence and/or the MR data, e.g. the image data reconstructed therefrom, has the same contrast. Accordingly, the recording of the long-term MR data typically takes place in a time-resolved manner within the first time period, wherein MR data mapping the region of interest is recorded for a plurality of time points within the first time period. The temporal sequence of the plurality of time points within the first time period determines the time-resolution of the long-term MR data.

The long-term MR data can comprise first long-term MR data and second long-term MR data, wherein the first long-term MR data was recorded with a first MR control sequence and/or a first contrast and the second long-term MR data with a second MR control sequence and/or a second contrast. The recording of the first long-term MR data and the second first long-term MR data may take place quasi-continuously and/or interleaved in each case.

If the long-term MR data is provided as raw data, the ascertainment of the first position data typically comprises a reconstruction of the long-term MR data into image data. The long-term MR data is typically slice image data and/or three-dimensional image data mapping the tissue. The first position data typically characterizes the tissue, e.g. a spatial extent and/or a shape (e.g. a shape of the surface), and/or a deformation of the tissue. The first position data is typically determined for at least two different first time points within the first time period for which at least two different first time points MR data was recorded in the context of the recording of the long-term MR data.

The first position data is time-resolved, wherein the time-resolution of the first position data typically corresponds to the time-resolution of the long-term MR data. The first position data is time-resolved and accordingly comprises first position data for at least two first time points that are different from one another. The at least two first time points that are different from one another typically correspond to at least one subset of the time points at which time points long-term MR data was recorded. The time-resolution of the long-term MR data can correspond to the time-resolution of the first position data. The time-resolution of the first position data can be less than the time-resolution of the long-term MR data. The ascertainment of the time-resolved first position data may comprise identification of the tissue in the long-term MR data and/or the image data reconstructed therefrom. The ascertainment of the time-resolved first position data may comprise segmentation of the tissue based on the long-term MR data. The ascertainment of the time-resolved first position data can also comprise registration of the tissue for different first time points.

The surface points are typically fixed points on the surface of the examination object. A surface point is typically a visually recognizable feature of the examination object, e.g. characterizing an extremity and/or an organ. A surface point can be a landmark representative of an extremity and/or an organ. The surface point can be indicated by a marker. The surface point can e.g. comprise a marker. The marker can, for example, be fixed, e.g. detachably fixed, on the surface and/or skin and/or clothing of the examination object. The surface point can be embodied as a marker, e.g. as a non-detachable marker, on a skin-tight item of clothing, e.g. an item of clothing that fits tightly to the examination object. As an example, the item of clothing can have been worn by the examination object for the purpose of acquiring the second position data in the first time period.

The second position data e.g. comprises position values for at least two second time points within the first time period for at least two surface points of the surface of the examination object. The position values of the at least two surface points can indicate the position of a surface point relative to a fixed point in the space, which space surrounds the examination object in the first time period. One of the at least two surface points can serve as a reference point, the position value of which is determined absolutely with reference to a fixed point in the space. At least one other position value for one of the at least two surface points can be determined relative to the reference point.

In the first time period, the long-term MR data and the second position data is acquired. The examination object is typically arranged within a magnetic resonance device in the first time period. The time-resolution of the long-term MR data, e.g. the time-resolution of the first position data, and the time-resolution of the second position data can be the same. The time-resolution of the first position data and the time-resolution of the second position data can be different from one another. The first position data comprises position data describing the tissue during at least two first time points within the first time period. The second position data comprises position data describing at least two surface points during at least two second time points within the first time period.

The number of second time points e.g. corresponds to the number of first time points. The first time points e.g. correspond to the second time points. On average, the first time points typically differ from the second time points by any suitable time period, such as for instance less than a minute, by less than ten seconds, by less than one second, etc. The correlation of the first position data with the second position data typically comprises the assignment of the first position data to the second position data taking account of the first time points and the second time points. The correlation typically takes place such that the first position data is assigned to the second position data such that the associated first time points and second time points differ minimally from one another.

The deformation model comprises the second position data dependent upon the first position data. Accordingly, the deformation model comprises a dependence of a shape and/or position of a tissue on an externally recognizable positioning of the examination object based on the surface of the examination object. The deformation model comprises the second position data dependent upon the first position data for e.g. at least two time points within the first time period. A movement of the examination object typically causes a change in the shape and/or position of the tissue and a change in the surface of the examination object. If the examination object moved within the first time period, e.g. between two first time points and/or two second time points, the deformation model comprises for at least two items of second position data, which are externally recognizable and correspond to a positioning of the examination object, an assignment to first position data characterizing a corresponding position and/or shape of the tissue.

The acquisition of the second position data from at least two points of a surface of the examination object and the acquisition of the long-term MR data e.g. takes place at least partially simultaneously in the first time period. The deformation model is typically specific to the examination object. The deformation model determined according to the disclosure comprises a particularly precise and individual correlation between features of the surface of the examination object that are externally recognizable without a magnetic resonance device and easy to acquire and details, e.g. a shape and/or position of the tissue, dependent upon the positioning of the examination object. Consequently, the deformation model determined according to the disclosure comprises a connection between an externally recognizable positioning of the examination object and a position and/or deformation of the tissue.

The use of long-term MR data enables the first position data to be determined particularly precisely since, e.g. in the case of lengthy magnetic resonance recordings, MR data with a particularly high quality and/or resolution can be generated, e.g. also for different positions of the examination object. The greater the number of different positions (e.g. positionings) the examination object is able to adopt in the first time period, the more precise and reliable the generation of the deformation model can be. Accordingly, the method according to the disclosure enables the generation of a particularly precise deformation model describing the shape and position of the tissue, in particular an organ, such as, for example, the liver, particularly precisely in dependence on externally recognizable features of the examination object. Consequently, when using the deformation model generated according to the disclosure, a shape and/or position of the tissue can be determined particularly precisely based on an externally and/or superficially recognizable positioning of the examination object even without the use of a medical imaging device.

One embodiment of the method provides a sensor arranged on at least one surface point of the at least two surface points of the surface of the examination object in the first time period. The sensor can correspond to a marker. A marker of a surface point can comprise a sensor. The sensor can be implemented e.g. as an acceleration sensor and/or a gyroscopic sensor and/or a position sensor. In addition to the position values that are dependent upon the second time points, the second position data may comprise sensor data that is dependent on the second time points. Depending on the type of sensor used, the sensor data can, for example, comprise acceleration and/or movement data. Such second position data can be particularly precise. A deformation model determined with such second position data is thus particularly precise.

One embodiment of the method provides that the second position data from the at least two surface points of the surface is optically acquired during the first time period. An optical acquisition of a surface of the examination object is particularly simple to achieve. For instance, this can be achieved particularly simply and based on landmarks.

One embodiment of the method provides that the second position data from the at least two surface points of the surface was acquired by means of at least one camera during the first time period. The second position data can also be acquired using two or more cameras. The camera can be implemented e.g. as a 3D camera and/or as a thermal imaging camera. Such cameras are particularly favorable. Similarly, the second position data can be acquired e.g. simply by means of a camera since it is based on landmarks and/or free of markers and/or sensors.

One embodiment of the method provides that at least one surface point of the at least two surface points of the surface of the examination object corresponds to one of the following landmarks: forehead, chin, nose, shoulder, elbow, knee, front of foot, heel, hip, wrist, skullcap, etc. A surface point of the at least two surface points can serve as a reference point relative to which reference point the remaining surface points can be determined. In particular the forehead and/or the skullcap can serve as a reference point. Said landmarks can be clearly identified optically and accordingly can be observed robustly in the first time period. Typically, these surface points are also simple to identify optically and/or with a camera and to track on a change of a positioning. A change in a position of one of said landmarks typically accurately represents a repositioning of the examination object. Accordingly, this embodiment of the method enables a particularly robust determination of the deformation model.

One embodiment of the method provides that the ascertainment of the time-resolved first position data comprises a determination of a fixed tissue point and/or tissue-specific landmarks. For instance, the ascertainment of time-resolved first position data may comprise segmentation of the tissue from the long-term MR data. The segmented tissue can, for example, be used as the basis for creating a three-dimensional model of the tissue for each first time point. A tissue-specific landmark can be a point on the surface and/or a middle point of the tissue. A tissue-specific landmark can also be a center of symmetry of the tissue. For instance, when using a three-dimensional model of the tissue, it is possible to determine a tissue-specific landmark particularly precisely. The first position data e.g. comprises position values for the tissue-specific landmarks. The position values of the tissue-specific landmarks are e.g. determined relative to a fixed tissue point, typically a particularly significant tissue-specific landmark. This enables a particularly precise determination of the first position data and thus a particularly precise deformation model.

One embodiment of the method provides that the correlation of the first position data with the second position data comprises relating the fixed tissue point to at least one surface point of the at least two surface points of the surface of the examination object.

As an example, the correlation of the first position data with the second position data comprises relating the fixed tissue point to the reference point. The correlation of the first position data with the second position data can also comprise an assignment of the fixed tissue point to one of at least any suitable number of surface points next to the fixed tissue point (e.g. at least 5, 3, 2, etc.). Relating the fixed tissue point to a surface point typically comprises a vector describing the distance between the fixed tissue point and the surface point and/or coordinates of the fixed tissue point relative to the surface point. According to this embodiment, the first position data typically comprises tissue-specific landmarks, which are indicated relative to the fixed tissue point. Relating the fixed tissue point to a surface point enables the deformation model to be determined particularly simply.

One embodiment of the method provides that the ascertainment of the time-resolved first position data comprises a determination of a statistical tissue model based on the tissue-specific landmarks. A statistical tissue model typically comprises an approximation of the shape of the tissue by at least one geometrical shape. The approximation typically comprises a determination of at least two centers and at least two geometric shapes, wherein in each case a geometric shape is arranged around a center and the size of the geometric shape is selected such that the shape of the tissue is approximated by the sum of the geometric shapes. Suitable geometric shapes are, for example, circles or ellipses. This embodiment enables a good approximation of the shape of the tissue without segmentation. Accordingly, this ascertainment of the time-resolved first position data can be performed particularly simply and without high computing effort. Alternatively and/or additionally, the statistical tissue model can be used for segmentation of the tissue.

One embodiment of the method provides that the ascertainment of the time-resolved first position data comprises the determination of a deformation field and/or a vector field based on the tissue-specific landmarks. The vector field is e.g. determined by the position values of the fixed tissue point at two successive first time points. In this embodiment, the position values e.g. comprise coordinates relative to a fixed point in the space. The vector field can be determined based on the position values of the fixed tissue point for each first time point relative to the preceding first time point. The vector field can analogously also be determined for each tissue-specific landmark. The deformation field e.g. comprises coordinates for at least one tissue-specific landmark, e.g. at least three tissue-specific landmarks, relative to the fixed tissue point. The deformation field and/or the vector field is e.g. determined for each first time point relative to the preceding first time point. This enables a precise description of a translation, i.e. a movement, and deformation of the tissue at each first time point. Accordingly, the tissue can be tracked particularly precisely. For instance, the deformation field and/or the vector field can correspond to a tissue model which is comprised by the first position data and describes the tissue at each first time point. In combination with the second position data, it is possible to generate a particularly detailed deformation model based on the deformation field and/or the vector field.

One embodiment of the method provides that the examination object adopted at least two positions during the first time period. The examination object is e.g. positioned differently during at least two of the first time points. The examination object statically adopts a first positioning during at least a first one of the first time points. The examination object statically adopts a second positioning during at least a second one of the first time points. The first positioning and/or the second positioning can also be quasi-static. Quasi-static positioning can be characterized in that during the quasi-static positioning, the examination object is subject to a movement, e.g. due to a physiological process, such as, for example breathing and/or a heartbeat. The examination object typically adopts the first positioning and the second positioning for any suitable time period, such as for example at least four minutes, at least ten minutes, at least twenty minutes, etc.

MR data, e.g. long-term MR data, may be recorded in each of the at least two positions. For example, a first positioning of the at least two positions can comprise the examination object lying in a lateral position. A second positioning of the at least two positions can, for example, comprise the examination object lying on the back and/or on the stomach. A third positioning of the at least two positions can, for example, comprise the examination object with bent and/or outstretched arms and/or legs. Depending on the difference between the at least two positions, it may be necessary to adapt the region of interest during the recording of the long-term MR data. Thus, during the first positioning, the tissue may be comprised by the region of interest, wherein during the second positioning the tissue would lie outside the region of interest, provided that there is no adaptation of the region of interest to the changed positioning of the examination object during the acquisition of the long-term MR data. The acquisition of the long-term MR data e.g. comprises an adaptation of the region of interest to a positioning of the examination object, e.g. dependent upon a position of the tissue in dependence upon a positioning of the examination object so that the adapted region of interest comprises the tissue. The adaptation of the region of interest can take place based on the second position data and/or based on at least one position of a surface point of a surface of the examination object.

The position and the shape of the tissue, which can be identified and/or quantitatively acquired based on the long-term MR data and/or e.g. the first position data, is dependent on the positioning of the examination object. If the long-term MR data and the second position data comprise information for at least two positions of the examination object that are different from one another, the deformation model can be determined more precisely since in particular further properties of the tissue are known. Similarly, it is conceivable that properties such as, for example, flexibility and/or elasticity, e.g. spatially-resolved elasticity, of the tissue can be determined on the basis of the first position data. Within the first time period, the examination object preferably adopts a plurality of different positions so that the position data of the tissue is known for all conceivable positions in dependence on the positioning of the examination object which is simple to determine on the basis of the surface points. A deformation model determined in this way is particularly versatile and enriches the digital twin.

One embodiment of the method provides that the first time period comprises at least one sleeping phase of the examination object. The first time period can comprise a nighttime time period. The recording of long-term MR data during a sleeping phase is particularly advantageous since, during sleep, the examination object adopts different natural positions and/or adopts different positions quasi-statically for a sufficiently long time period for the recording of MR data. In particular, sleep typically takes place in a horizontal position, which is compatible with a positioning in a conventional magnetic resonance device. As an example, the acquisition of the long-term MR data can take place by means of a dedicated magnetic resonance device. The dedicated magnetic resonance device can be specifically adapted to the requirements while a patient is sleeping such as, for example, a patient-receiving region of above-average size of at least 75 cm, at least 85 cm, etc., and/or function at least partially free of local receiving coils.

One embodiment of the method provides that the correlation of the first position data with the second position data comprises training an artificial neural network. The training of the artificial neural network is e.g. specific to the examination object. The artificial neural network e.g. comprises an input layer, an output layer and/or any suitable number of layers (e.g. at least 100 layers). The training of the artificial neural network e.g. takes place by means of deep learning. Input data provided to the input layer in the context of the training typically comprises the second position data, e.g. during at least two first time points. Output data provided to the output layer in the context of the training typically comprises the first position data, e.g. during at least two first time points. The training of the artificial neural network e.g. comprises augmentation of the data, e.g. the input data, and/or a dropout. The training of the artificial neural network can comprise an adaptation of the increment of the time-resolution of the first position data and/or the second position data. The training of the artificial neural network enables the deformation model to be extended to at least one further positioning of the examination object for which further positioning no first position data is available and/or which further positioning the examination object had not adopted in the first time period. A deformation model determined in this way enables a more comprehensive prediction for an extended number of positions of the examination object. This increases the precision of the deformation model. It is also conceivable for the artificial neural network at least partially to comprise information from a training object that is different from the examination object and/or for the artificial neural network to be individualized on the basis of the described training for the examination object.

One embodiment of the method provides that the deformation model is stored as part of a “digital twin” of the examination object. The digital twin of an examination object corresponds to its virtual image comprising all medical data, for example medical image data and diseases and the resulting metadata. A deformation model of a tissue enriches numerous applications and can, for example, be used in the context of multimodality examinations, e.g. cross-modality imaging methods. The long-term MR data is e.g. also stored as part of the digital twin.

Furthermore, the disclosure is based on a method for determining third position data describing a tissue in dependence on a positioning of an examination object in accordance with the following method steps:

ascertaining of fourth position data from at least one surface point of a surface of the examination object;

provisioning a deformation model generated according to the method according to the disclosure for the examination object for the tissue; and

determining third position data describing the tissue based on the fourth position data and the deformation model.

The fourth position data is typically ascertained based on at least two surface points of the surface of the examination object. For instance, during the ascertainment of the fourth position data, the surface points of the surface of the examination object correspond to the surface points of the surface of the examination object in the first time period. The fourth position data is e.g. ascertained analogously to the second position data. The fourth position data is, for example, acquired optically and/or using a camera and/or using sensors arranged on the surface points. The fourth position data is typically acquired without using a magnetic resonance device. Accordingly, this method enables efficient use of the deformation model generated according to the disclosure as a result of which it is possible to determine a shape and position of the tissue based on the externally identifiable fourth position data.

One embodiment of the method provides the use of the third position data in the context of an interventional examination of the examination object and/or in combination with image data mapping the tissue of the examination object. The fourth position data is acquired in the second time period. The second time period occurs after the completion of the first time period and/or after the generation of the deformation model. In the second time period, it is also possible for image data to be acquired from a region of interest comprising the tissue of the examination object using a further medical imaging device. An interventional procedure can also take place in the second time period, wherein, for example for the navigation of a catheter, the third position data is used in combination with image data generated using the further medical imaging device in the second time period. Additionally, the image data generated in the second time period can be combined with the long-term MR data to improve the quality of the image data generated in the second time period.

The further medical imaging device can, for example, comprise an ultrasound device and/or an angiography device and/or an X-ray device. The further medical imaging device can comprise a second magnetic resonance device, which second magnetic resonance device e.g. comprises a main magnet that generates a lower main magnetic field than the main magnet comprised by the magnetic resonance device, which magnetic resonance device was used to acquire the long-term MR data from the examination object. This is particularly advantageous since a lower main magnetic field causes less interaction with the catheter and/or further interventional material.

The third position data is determined in the second time period e.g. in real time and/or dynamically. The third position data can be registered, e.g. dynamically registered, with the image data of the tissue to be recorded in the second time period in real time. After registration, the third position data can be displayed superimposed on the tissue. Similarly, the third position data can be used as the basis for determining the volume and/or the shape of the tissue, which can be depicted superimposed on the image data.

The deformation model generated according to the disclosure in particular enables a dynamic determination of a shape and position of the tissue based on externally recognizable surface points.

Similarly, the third position data can be used to register the image data recorded with a further medical imaging device with the long-term MR data particularly precisely, in particular in the region of the tissue. This is particularly advantageous for hybrid imaging devices.

Furthermore, the disclosure is based on a further method for generating a deformation model for a tissue of an examination object dependent upon a positioning of the examination object in accordance with the following method steps:

acquiring long-term MR data from a region of interest comprising the tissue of the examination object in a first time period using a magnetic resonance device;

ascertaining time-resolved first position data describing the tissue based on the long-term MR data;

acquiring time-resolved second position data from at least two surface points of a surface of the examination object in the first time period using a detector unit; and

determining the deformation model for the tissue by correlating the first position data with the second position data.

One embodiment of the method provides that in each case a sensor is arranged on the at least two surface points of the surface of the examination object in the first time period. One embodiment of the method provides that the acquisition of the time-resolved second position data during the first time period takes place optically and/or by means of a camera. One embodiment of the method provides that the first time period comprises at least one sleeping phase of the examination object. One embodiment of the method provides that the examination object adopts at least two positions during the first time period.

Embodiments of the further method according to the disclosure are analogous to the embodiments of the aforementioned method according to the disclosure. Similarly, features, advantages, or alternative embodiments mentioned here can also be transferred to the further method(s), and vice versa.

The disclosure is further based on a system, comprising:

a first input configured to acquire long-term MR data recorded in a first time period from a region of interest comprising the tissue of the examination object;

an ascertaining unit configured to ascertain time-resolved first position data describing the tissue based on the long-term MR data;

a second input configured to acquire time-resolved second position data recorded in the first time period from at least two surface points of the surface of the examination object; and

a determining unit configured to determine the deformation model for the tissue by correlating the first position data with the second position data. The system is configured or otherwise implemented to carry out a method according to the disclosure for generating a deformation model for a tissue of an examination object dependent upon a positioning of the examination object.

For this, the ascertaining unit is typically connected to the first input. The determining unit is typically connected to the second input. The ascertaining unit and/or the determining unit typically has an input, a processor unit, and an output. Via the input, the ascertaining unit can, for example, be provided with the long-term MR data. Via the input, the determining unit can, for example, be provided with the second position data. Further functions, algorithms, artificial neural networks, or parameters required in the method can be provided to the determining unit and/or the ascertaining unit via the input. The deformation model and/or further results of an embodiment of the method according to the disclosure can be provided via the output.

The disclosure is further based on a magnetic resonance device comprising a system according to the disclosure, an ascertaining unit, which ascertaining unit is configured to record long-term MR data from a region of interest of an examination object, and a detector unit, which detector unit is configured to acquire position data from at least two surface points of a surface of the examination object. The ascertaining unit typically comprises a main magnet, a gradient coil unit, and a radio-frequency antenna unit surrounding a patient-receiving region. The detector unit can be implemented as a camera, e.g. a 3D-camera and/or a thermal imaging camera. The system can be integrated in the magnetic resonance device. The system can also be installed separately from the magnetic resonance device. The system can be connected to the magnetic resonance device. The magnetic resonance device is configured to carry out a method according to the disclosure for generating a deformation model for a tissue of an examination object dependent upon a positioning of the examination object. The magnetic resonance device is in e.g. configured to carry out the further method according to the disclosure for generating a deformation model for a tissue of an examination object in dependence on a positioning of the examination object, including the acquisition of long-term MR data and the acquisition of time-resolved second position data.

Embodiments of the system according to the disclosure and of the magnetic resonance device according to the disclosure are embodied analogously to the embodiments of the method according to the disclosure. The magnetic resonance device and/or the system can have further control components which are necessary and/or advantageous for carrying out method(s) according to the disclosure. The magnetic resonance device can also be configured to send control signals and/or to receive control signals and/or to process them to execute method(s) according to the disclosure. The determining unit and/or the ascertaining unit may e.g. be part of a generating unit of the system according to the disclosure. The determining unit and/or the ascertaining unit e.g. form a generating unit. The determining unit and/or the ascertaining unit can also be comprised by the generating unit. A memory unit (e.g. a non-transitory computer-readable medium) of the generating unit can be used to store computer programs and further software and/or an artificial neural network, by means of which the processor unit of the generating unit may execute to automatically control and/or perform the method sequence of one or more methods according to the disclosure.

A computer program product according to the disclosure can be loaded directly into a memory unit of a programmable generating unit and has program code means for carrying out one or more methods according to the disclosure when the computer program product is executed in the generating unit. This enables the one or more methods according to the disclosure to be carried out quickly, identically repeatedly, and robustly. The computer program product is configured such that it is able to carry out the method steps according to the disclosure by means of the generating unit. Herein, the generating unit in each case may have relevant prerequisites such as, for example, a corresponding working memory, a corresponding graphics cards or a corresponding logic unit, etc., so that the respective method steps can be carried out efficiently. The computer program product is, for example, stored on an electronically readable medium or on a network or server from where it can be loaded into the processor of a local generating unit, which can be connected directly to the system and/or magnetic resonance device or embodied as part of the system and/or magnetic resonance device. Furthermore, control information of the computer program product can be stored on an electronically readable data carrier. The control information of the electronically readable data carrier can be designed such that that it carries out one or more methods according to the disclosure when the data carrier is used in a generating unit of a system. Examples of electronically readable data carriers are a DVD, a magnetic tape or a USB stick on which electronically readable control information, in particular software, is stored. When this control information (software) is read from the data carrier and stored in a generating unit of a system, all the embodiments according to the disclosure of the above-described method can be carried out.

Furthermore, the disclosure is based on an electronically readable data carrier holding a program that is provided to carry out a method for generating a deformation model for a tissue of an examination object in dependence on a positioning of the examination object.

The advantages of the system according to the disclosure, the magnetic resonance device according to the disclosure, the computer program product according to the disclosure, and the electronically readable data carrier according to the disclosure substantially correspond to the advantages of the method according to the disclosure for generating a deformation model for a tissue of an examination object in dependence on a positioning of the examination object which were explained in detail above. Features, advantages, or alternative embodiments can similarly also be transferred to the other claimed subject matter, and vice versa.

BRIEF DESCRIPTION OF THE DRAWINGS/FIGURES

Further advantages, features and details of the disclosure may be derived from the exemplary embodiments described below and with reference to the drawings, which show:

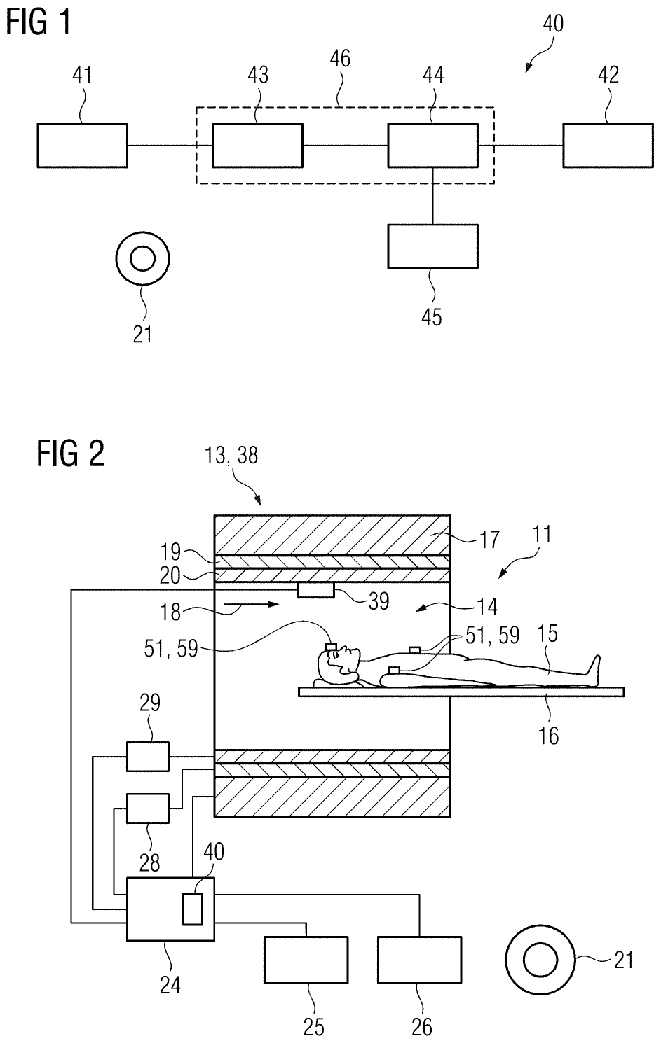

FIG. 1 illustrates a system in a schematic depiction according to one or more embodiments of the present disclosure;

FIG. 2 illustrates a magnetic resonance device in a schematic depiction according to one or more embodiments of the present disclosure;

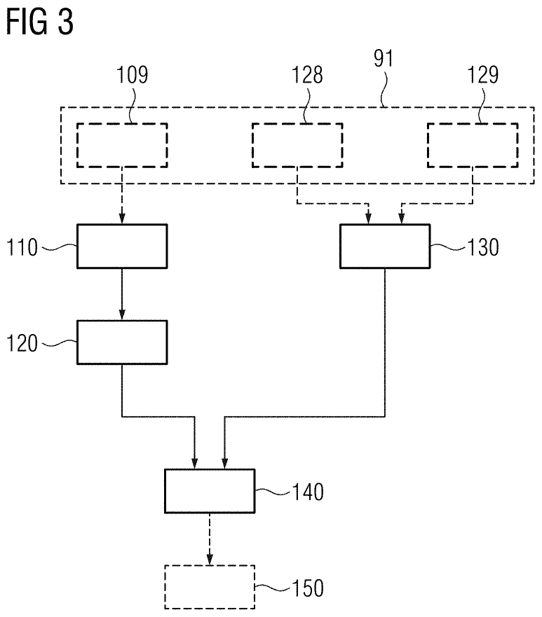

FIG. 3 illustrates a flow diagram of a first embodiment of a method according to the disclosure;

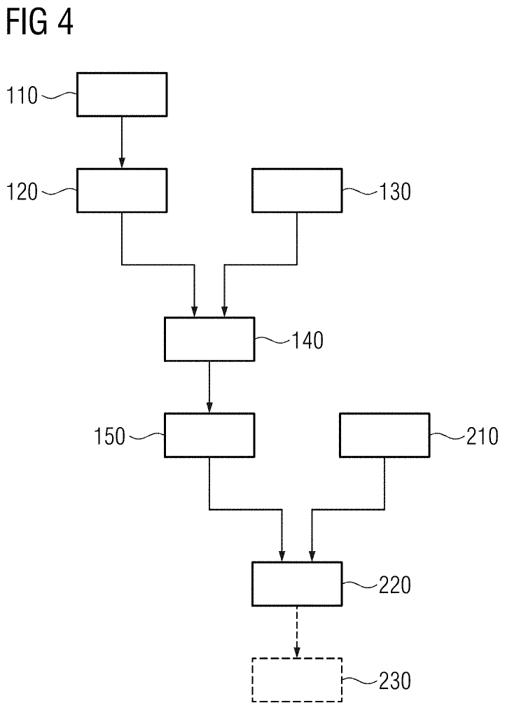

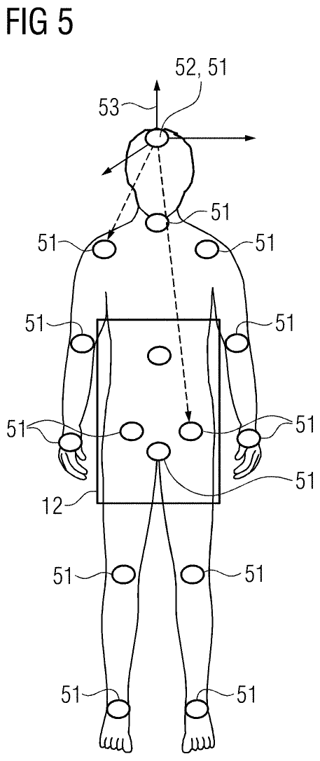

FIG. 4 illustrates a flow diagram of a second embodiment of a method according to the disclosure;

FIG. 5 illustrates an examination object in a schematic depiction according to one or more embodiments of the present disclosure;

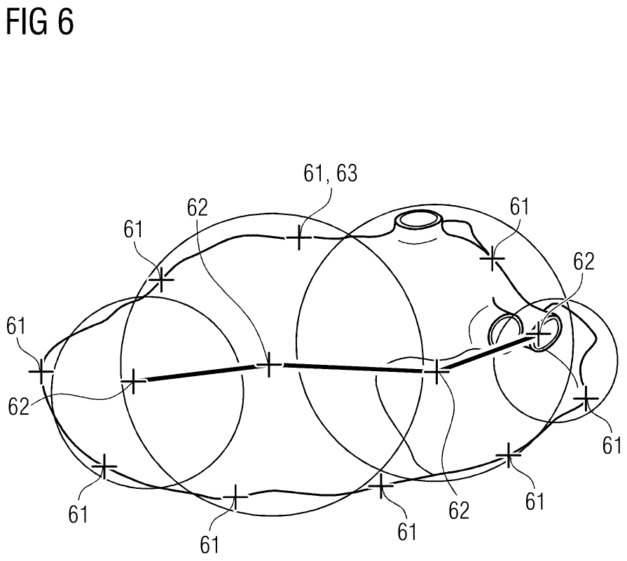

FIG. 6 illustrates a heart in a schematic depiction including first position data at a first time point according to one or more embodiments of the present disclosure; and

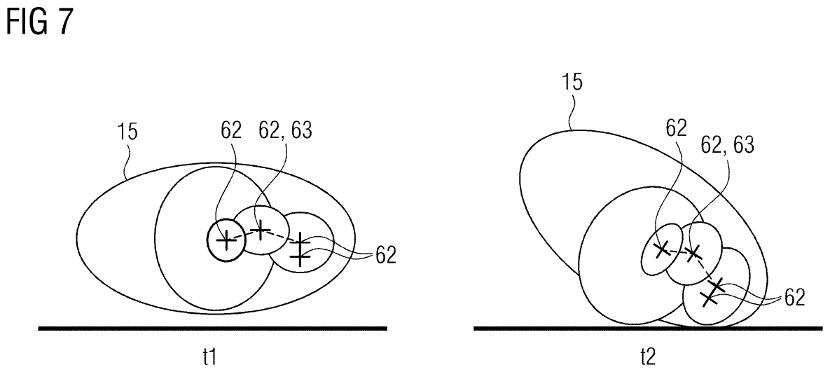

FIG. 7 illustrates two positions of the examination object that are different from one another in a schematic depiction according to one or more embodiments of the present disclosure.

### DETAILED DESCRIPTION

FIG. 1 shows a system 40 according to an embodiment of the disclosure for carrying out a method according to the disclosure in a schematic depiction. The system 40 comprises a first input 41 configured to acquire long-term MR data recorded in a first time period 91 from a region of interest 12 comprising the tissue of the examination object 15. The system 40 comprises an ascertaining unit 43 configured to ascertain time-resolved first position data describing the at least one tissue based on the long-term MR data. The system 40 comprises a second input 42 configured to acquire time-resolved second position data recorded in the first time period 91 from at least two surface points 51 of the surface of the examination object 15. The system 40 comprises a determining unit 44 configured to determine the deformation model for the tissue by correlating the first position data with the second position data. The first input 41 is e.g. connected to the ascertaining unit 43 and/or integrated therein. The second input 42 is e.g. connected to the determining unit 44 and/or integrated therein. The determining unit 44 and the ascertaining unit 43 can be part of a generating unit 46. The determining unit 44 and the ascertaining unit 43 can be implemented as separate from one another or as a single unit. The system 40 e.g. comprises an output 45 via which the system 40 can output the deformation model generated. The output 45 is typically connected to the determining unit 44.

For this, the determining unit 44 and/or the ascertaining unit 43, e.g. the generating unit 46, have computer programs and/or software, which can be loaded directly into a memory unit (and which are not depicted in further detail) of the determining unit 44 and/or ascertaining unit 43 and/or generating unit 46, with program means for carrying out a method for generating a deformation model for a tissue of an examination object 15 in dependence on a positioning of the examination object 15 when the computer programs and/or software are executed in the determining unit 44 and/or ascertaining unit 43 and/or generating unit 46. For this, the determining unit 44 and/or ascertaining unit 43 and/or generating unit 46 may have one or more processors, processing circuitry, etc. not depicted in further detail, configured or otherwise designed to execute the computer programs and/or software. Alternatively thereto, the computer programs and/or software can also be stored on an electronically readable data carrier 21 embodied separately from the determining unit 44 and/or ascertaining unit 43 and/or generating unit 46, wherein the determining unit 44 and/or ascertaining unit 43 and/or generating unit 46 can have data access to the electronically readable data carrier 21 via a data network. The system 40 typically comprises a display unit and/or an input unit.

The depicted system 40 can comprise further components. Hence, the system 40 is configured, together with the determining unit 44 and the ascertaining unit 43, to carry out a method according to the disclosure for generating a deformation model for a tissue of an examination object 15 dependent upon a positioning of the examination object 15.

A method for generating a deformation model for a tissue of an examination object 15 dependent upon a positioning of the examination object 15 can also be provided in the form of a computer program product that implements the method on the system 40 and/or on the generating unit 46 when it is carried out on the system 40 and/or on the generating unit 46. Similarly, an electronically readable data carrier 21 can be provided with electronically readable control information, which comprises at least one such above-described computer program product and is designed to carry out the described method when the data carrier 21 is used in a determining unit 44 and/or ascertaining unit 43 and/or generating unit 46 of a system 40.

FIG. 2 shows a magnetic resonance device 11 according to the disclosure for carrying out a method according to the disclosure and embodied to record long-term MR data in a schematic depiction. The magnetic resonance device 11 comprises an ascertaining unit 38 formed by a magnet unit 13 with a main magnet 17 for generating a strong and constant main magnetic field 18. The magnetic resonance device 11 also has a cylindrical patient-receiving region 14 for receiving an examination object 15, wherein the patient-receiving region 14 is enclosed by the magnet unit 13 in a cylindrical shape in a circumferential direction. The examination object 15 can be pushed into the patient-receiving region 14 by means of a patient-support apparatus 16 of the magnetic resonance device 11. For this, the patient-support apparatus 16 has a patient table arranged movably within the magnetic resonance device 11.

The magnet unit 13 also has a gradient coil unit 19 used for position encoding during imaging. The gradient coil unit 19 is actuated by means of a gradient control unit 28. Furthermore, the magnet unit 13 has a radio-frequency antenna unit 20, which, in the case shown, is implemented as a body coil permanently integrated in the magnetic resonance device 11, and a radio-frequency antenna control unit 29 for exciting polarization that is established in the main magnetic field 18 generated by the main magnet 17. The radio-frequency antenna unit 20 is actuated by the radio-frequency antenna control unit 29 and radiates high-frequency radio-frequency pulses into an examination space, which is substantially formed by the patient-receiving region 14.

To control the main magnet 17, the gradient control unit 28, and the radio-frequency antenna control unit 29, the magnetic resonance device 11 has a control unit 24. The control unit 24 centrally controls the magnetic resonance device 11, such as, for example, the performance of MR control sequences. The control unit 24 also comprises a reconstruction unit, not depicted in further detail, for reconstructing medical image data acquired during the magnetic resonance examination. The magnetic resonance device 11 has a display unit 25. Control information such as, for example, control parameters and reconstructed image data can be displayed on the display unit 25, for example on at least one monitor, for a user.

The magnetic resonance device 11 also has an input unit 26 by means of which a user can input information and/or control parameters during a scanning process. The control unit 24 can comprise the gradient control unit 28 and/or radio-frequency antenna control unit 29 and/or the display unit 25 and/or the input unit 26. As a result, the magnetic resonance device 11 is configured to acquire long-term MR data from a region of interest 12 comprising the tissue of the examination object 15. For this, the control unit 24 has computer programs and/or software, which can be loaded directly into a memory unit of the control unit 24, not depicted in further detail, with program means for carrying out a method for recording long-term MR data of the examination object 15 when the computer programs and/or software are executed in the control unit 24. For this, the control unit 24 has a processor, not depicted in further detail, designed to execute the computer programs and/or software.

The magnetic resonance device 11 comprises a detector unit 39 configured to acquire time-resolved second position data from at least two surface points 51 of a surface of the examination object 15. In each case, a sensor 59 is e.g. arranged on the at least two surface points 51 of the surface of the examination object 15. The magnetic resonance device 11 comprises a system 40 according to the disclosure the individual components of which can be seen in FIG. 1. The system 40 is typically connected to the detector unit 39 and the ascertaining unit 38. The system 40 can be integrated in the control unit 24. The system 40 can be arranged and/or designed as separate from the control unit 24. The system 40 can e.g. be operated by means of the display unit 25 and/or the input unit 26.

The magnetic resonance device 11 depicted can obviously comprise further components that magnetic resonance devices 11 normally have. In addition, the general mode of operation of a magnetic resonance device 11 is known to the person skilled in the art, so there is no detailed description of the further components. Hence, the magnetic resonance device 11 is configured to carry out a method according to the disclosure.

The magnetic resonance device 11 according to the disclosure is e.g. also configured to carry out a further method for generating a deformation model for a tissue of an examination object 15 dependent upon a positioning of the examination object 15 in accordance with the following method steps:

acquiring long-term MR data from a region of interest 12 comprising the tissue of the examination object 15 in a first time period 91 using a magnetic resonance device 11;

ascertaining time-resolved first position data describing the tissue based on the long-term MR data;

acquiring time-resolved second position data from at least two surface points 51 of a surface of the examination object 15 in the first time period 91 using a detector unit 39; and

determining the deformation model for the tissue by correlating the first position data with the second position data.

The further method for generating a deformation model can also be provided in the form of a computer program product that implements the method on the control unit 24 when it is executed on the control unit 24. Similarly, an electronically readable data carrier 21 can be provided with electronically readable control information stored thereupon, which comprises at least one such above-described computer program product and is designed to carry out the described further method when the data carrier 21 is used in a control unit 24 of a magnetic resonance device 11.

FIG. 3 shows a flow diagram of a first embodiment of a method according to the disclosure for generating a deformation model for a tissue of an examination object 15 dependent upon a positioning of the examination object 15. At the start of the method, the provisioning of long-term MR data recorded in a first time period 91 from a region of interest 12 comprising the tissue of the examination object 15 takes place in method step 110. When method step 110 has been carried out, the ascertainment of time-resolved first position data describing the tissue based on the long-term MR data takes place in method step 120. The provisioning of time-resolved second position data recorded in the first time period 91 from at least two surface points 51 of a surface of the examination object 15 takes place in accordance with method step 130, e.g. independently of method steps 110 and 120. When method steps 130 and 120 have been carried out, the determination of the deformation model for the tissue by correlating the first position data with the second position data takes place in accordance with method step 140. The deformation model determined can optionally be provided in accordance with method step 150. For instance, the provision in accordance with method step 150 can be stored as part of a digital twin of the examination object 15.

Method step 130 can optionally be preceded by method step 128 and/or method step 129 during the first time period 91. According to method step 128, the second position data from the at least two surface points 51 of the surface can be optically acquired during the first time period 91. According to method step 129, the second position data from the at least two surface points 51 of the surface can be acquired by means of a camera during the first time period 91. According to method steps 128 and/or 129, the second position data from the at least two surface points 51 of the surface can be acquired by means of the detector unit 39 during the first time period 91. Method step 110 can optionally be proceeded by method step 109 during the first time period 91. In accordance with method step 109, long-term MR data can be acquired from the region of interest 12 comprising the tissue of the examination object 15 by means of a magnetic resonance device 11, in particular by means of an ascertaining unit 38.

FIG. 4 shows a flow diagram of a second embodiment of a method according to the disclosure. With respect to method steps 110, 120, 130, 140, 150, reference is made to the description of FIG. 3. In addition, the second embodiment of the method according to the disclosure for determining third position data describing a tissue dependent upon a positioning of the examination object 15 and provides the ascertainment of fourth position data from at least one surface point 51 of a surface of the examination object 15 in method step 210. According to method step 220, the determination of third position data describing the tissue takes place based on the fourth position data and the deformation model. Optionally, according to method step 230, the use of the third position data can take place in the context of an interventional examination of the examination object 15 and/or in combination with image data mapping the tissue of the examination object 15.

FIG. 5 shows an examination object 15 in a schematic depiction. The at least two surface points 51 of the surface of the examination object 15, from which at least two surface points 51 second position data is recorded in the first time period 91, are depicted and each correspond to one of the following landmarks: forehead, chin, nose, shoulder, elbow, knee, front of foot, heel, hip, wrist, skullcap. However, these surface points are provided by way of example and not limitation, and additional or alternate surface points 51 may be used. In an embodiment, in each case a sensor 59 is arranged on the at least two surface points 51. One of the at least two surface points 51, e.g. the surface point 51 corresponding to the skullcap, can define a reference point 52.

The second position data can e.g. comprise the relative positions of the at least two surface points 51 to the reference point 52. Similarly, the reference point 52 can define a reference coordinate system 53. The reference coordinate system 53 is typically aligned along an anatomical axis of the examination object 15, in particular the skull of the examination object 15.

FIG. 6 shows a heart in a schematic depiction including first position data at a first time point. The heart is an example of an organ as a tissue of the examination object 15. The first position data at the first time point, which is ascertained in method step 120 based on the long-term MR data, e.g. comprises tissue-specific landmarks 61, 62. These tissue-specific landmarks 61, 62 can, for example, be differentiated as surface landmarks 61, which are, for example, arranged on a surface of the heart, and middle-point landmarks 62, defined as landmarks arranged in the middle between two surface landmarks 61. Similarly, it is possible to use a dedicated tissue-specific landmark 61, 62 as a fixed tissue point 63. During the correlation of the first position data with the second position data in accordance with method step 140, there is e.g. a determination of the relationship between the fixed tissue point 63 and at least one of the at least two surface points 51. The fixed tissue point 63 is typically comprised by the region of interest 12. The one of the at least two surface points 51 can lie outside the region of interest 12.

The first position data can comprise a statistical tissue model based on the tissue-specific landmarks. FIG. 6 shows such a statistical tissue model at the first time point. The statistical tissue model can, for example, take place by adapting graphics primitives, such as, for example, spheres and/or ellipsoids, based on the middle-point landmarks 62 to the tissue. Herein, it is e.g. possible to use the surface landmarks 61 and/or the surface of the tissue as a restriction for the determination of the size of the graphics primitives.

The ascertainment of the time-resolved first position data in accordance with method step 120 can comprise a determination of a deformation field and/or a vector field based on the tissue-specific landmarks 61, 62. As an example, the fixed tissue point 63 can also be taken into account when determining the deformation field and/or the vector field. The tissue-specific landmarks 61, 62 and thus also the fixed tissue point 63 typically have positions that are different from one another during at least two first time points that are different from one another within the first time period 91.

The absolute position of the fixed tissue point 63 at two first time points that are different from one another, in particular two successive first time points, within the first time period 91 can be described as a vector field. Consequently, the vector field describes at least one translation of the fixed tissue point 63 within the first time period 91. Similarly, the fixed tissue point 63 can be used as the origin of a coordinate system relative to which all tissue-specific landmarks 61, 62 for at least two first time points within the first time period 91 are determined in the form of coordinates. Coordinates of the tissue-specific landmarks 61, 62 determined in this way correspond to a deformation field describing a deformation of the tissue.

FIG. 7 shows the examination object 15 within the first time period 91 in two positions that are different from one another at two time points t1, t2 in a schematic depiction. In the embodiment shown, the fixed tissue point 63 and all the tissue-specific landmarks depicted correspond to middle-point landmarks 62. A comparison of the tissue at both time points has a translation described by a change of the fixed tissue point 63, for example as a vector field, and a deformation described by the position of the tissue-specific landmarks 61, 62 relative to the fixed tissue point 63, for example as a deformation field. In the case depicted, the examination object 15 in each case adopts a lying position such as is, for example, possible during a sleeping phase of the examination object 15 at the time points t1, t2. The two time points t1, t2 may be, for instance, first time points and second time points. Although the disclosure was illustrated and described in further detail by the preferred exemplary embodiments, the disclosure is not restricted by the disclosed examples and other variations can be derived herefrom by the person skilled in the art without departing from the scope of protection of the disclosure.

What is claimed is:

1. A method for generating a deformation model for a tissue of an examination object dependent upon a positioning of the examination object, comprising:
acquiring, via one or more processors, magnetic resonance (MR) data recorded during a first time period from a region of interest comprising the tissue of the examination object;
ascertaining, via one or more processors, time-resolved first position data describing the tissue based on the MR data;
acquiring, via one or more processors, time-resolved second position data recorded during the first time period from at least two surface points associated with a surface of the examination object; and
determining, via one or more processors, the deformation model for the tissue by correlating the first position data with the second position data.

2. The method as claimed in claim 1, further comprising:
arranging a sensor on at least one surface point of the at least two surface points of the surface of the examination object during the first time period.

3. The method as claimed in claim 1,
wherein the act of acquiring the time-resolved second position data comprises optically acquiring the time-resolved second position data during the first time period.

4. The method as claimed in claim 1,
wherein the act of acquiring the time-resolved second position data comprises acquiring the second position data via a camera during the first time period.

5. The method as claimed in claim 1,
wherein at least one surface point of the at least two surface points of the surface of the examination object corresponds to one of the following landmarks:
a forehead, chin, nose, shoulder, elbow, knee, front of foot, heel, hip, wrist, or skullcap of the examination object.

6. The method as claimed in claim 1,
wherein the act of ascertaining the time-resolved first position data comprises determining a fixed tissue point.

7. The method as claimed in claim 1,
wherein the act of ascertaining the time-resolved first position data comprises determining tissue-specific landmarks.

8. The method as claimed in claim 7,
wherein the act of ascertaining the time-resolved first position data comprises determining a statistical tissue model based on the tissue-specific landmarks.

9. The method as claimed in claim 7,
wherein the act of ascertaining the time-resolved first position data comprises determining a deformation field and/or a vector field based on the tissue-specific landmarks.

10. The method as claimed in claim 6,
wherein the act of determining the deformation model comprises correlating the first position data with the second position data by relating the fixed tissue point with at least one surface point of the at least two surface points of the surface of the examination object.

11. The method as claimed in claim 1,
wherein the examination object adopts at least two positions during the first time period.

12. The method as claimed in claim 1,
wherein the first time period comprises at least one sleeping phase associated with the examination object.

13. The method as claimed in claim 1,
wherein the act of determining the deformation model comprises correlating the first position data with the second position data by using a trained artificial neural network.

14. The method as claimed in claim 1, further comprising:
storing the deformation model as part of a virtual image of the examination object.

15. The method as claimed in claim 1, further comprising:
ascertaining third position data from at least one surface point of the surface of the examination object; and
determining fourth position data describing the tissue based on the third position data and the deformation model.

16. The method as claimed in claim 15, further comprising:
using the fourth position data to perform an interventional examination of the examination object and/or in combination with image data mapping the tissue of the examination object.

17. The method as claimed in claim 1, wherein the first time period has a duration of at least one hour.

18. A non-transitory computer-readable medium having instructions stored thereon that, when executed by one or more processors, cause the one or more processors to generate a deformation model for a tissue of an examination object dependent upon a positioning of the examination object by:
acquiring magnetic resonance (MR) data recorded during a first time period from a region of interest comprising the tissue of the examination object;
ascertaining time-resolved first position data describing the tissue based on the MR data;
acquiring time-resolved second position data recorded during the first time period from at least two surface points associated with a surface of the examination object; and
determining the deformation model for the tissue by correlating the first position data with the second position data.

19. A magnetic resonance (MR) device for generating a deformation model for a tissue of an examination object in dependence on a positioning of the examination object, comprising:
a first input configured to acquire MR data recorded during a first time period from a region of interest comprising the tissue of the examination object;
ascertaining circuitry configured to ascertain time-resolved first position data describing the tissue based on the MR data;
a second input configured to acquire time-resolved second position data recorded during the first time period from at least two surface points of the surface of the examination object; and
determining circuitry configured to determine the deformation model for the tissue by correlating the first position data with the second position data.

20. The MR device of claim 19, further comprising:
detector circuitry configured to acquire the second position data from the at least two surface points of the surface of the examination object.
